(19) United States
(12) Patent Application Publication
(10) Pub. No.: US 2013/0096507 A1
(43) Pub. Date: Apr. 18, 2013

(54) MEDICAL APPLIANCE SECURING DEVICE AND METHOD OF USING THE SAME

(71) Applicant: Matthew J. LeLievre, Estero, FL (US)

(72) Inventor: Matthew J. LeLievre, Estero, FL (US)

(73) Assignee: M.C. JOHNSON CO., INC., Fort Myers, FL (US)

(21) Appl. No.: 13/536,469

(22) Filed: Jun. 28, 2012

Related U.S. Application Data
(60) Provisional application No. 61/571,276, filed on Jun. 28, 2011.
(60) Provisional application No. 61/575,078, filed on Aug. 15, 2011.

(51) Int. Cl. A61M 25/02

(52) U.S. Cl. CPC A61M 25/02
USPC 604/179

(57) ABSTRACT

Medical appliance securing device and method of using the same to secure a medical appliance to a patient. In one embodiment, the device may be used to secure a hemodialysis catheter to a patient and may include a first base portion, a first adhesive for securing the first base portion to an object, such as a patient, the first adhesive being positioned under and coupled to the first base portion, a second base portion, the second base portion being positionable over the first base portion and fastenable to the first base portion; and a strap, the strap having a first end coupled to the first base portion and a second end fastenable to the second base portion.

### CROSS-REFERENCE TO RELATED APPLICATIONS

The present application claims the benefit under 35 U.S.C. 119(e) of U. S. Provisional Patent Application Ser. No. 61/571,276, filed Jun. 28, 2011, and of U.S. Provisional Patent Application Ser. No. 61/575,078, filed Aug. 15, 2011, the disclosures of both of which are incorporated herein by reference.

### BACKGROUND OF THE INVENTION

The present invention relates generally to medical appliance securing devices and relates more particularly to a novel medical appliance securing device and to a method of using the same to secure a medical appliance to a patient.

Various medical devices, or portions thereof, are shaped as generally tubular members. Examples of such devices include, but are not limited to, catheters, catheter hubs, thermometers, intravenous needles, intravenous needle hubs, and various types of electrical wires. There is often a need to secure such devices to or near a patient's body so that the devices can appropriately function. For example, catheters, which are generally long, tubular, flexible conduits used to transport various types of fluids to and from the body of a patient, are often laterally and/or longitudinally fixed to the body of a patient to ensure proper placement and functioning of the catheter, as well as to offer some degree of comfort and mobility for the patient. In particular, some medical procedures involving the use of catheters require the application of a tensile force, known as traction, to the catheter, thus making it particularly necessary to firmly secure the catheter in its longitudinal direction.

To secure a catheter, a hub, or other medical appliance in the manner indicated above, practitioners have often used one or more strips of conventional, medical-grade, adhesive tape to secure a length of the medical appliance directly to the patient's skin. However, this method has its shortcomings as the adhesive tape tends to become loose over time. In addition, each time that one wishes to adjust the placement of the medical appliance or each time that one wishes to remove the medical appliance from the patient, the adhesive tape must be removed from the skin of the patient, thereby frequently causing irritation and discomfort to the patient. Moreover, where the medical appliance is an intravenous catheter or the like, it is important that the catheter be secured to the patient in such a way as to minimize side-to-side rolling movement of the catheter, particularly when the patient moves or when a feeding set or other medical appliance is connected to the catheter, typically via a hub located at the proximal end of the catheter. If such rolling movement is not minimized, the catheter may cause inflammation of the patient's vein (i.e., phlebitis). Typically, securement is attempted by applying a strip of tape over the catheter hub and the patient's skin. However, as can be appreciated, because the catheter hub is rounded, the strip of tape typically contacts the skin at points spaced away from the catheter hub, thereby enabling the catheter hub some freedom to roll. As can be appreciated, the less surface area contacted with the strip of tape, the more free the hub will be to roll.

One common type of medical catheter is a hemodialysis catheter, an example of which is shown in FIG. 1, said hemodialysis catheter being represented generally by reference numeral 11. Hemodialysis catheter 11 comprises a length of tubing 13 having a distal end 15 that is adapted to be inserted into a vein of a patient and a proximal end 17 that is coupled to a generally cone-shaped hub 19. Tubing 13 has a pair of longitudinal lumens (not shown), one of which is fluidly coupled through a channel (not shown) in hub 19 to a length of tubing 21 and the other of which is fluidly coupled through a channel (not shown) in hub 19 to a length of tubing 23. In use, distal end 15 is introduced, using a needle or other access device (not shown), into a vein near the collarbone of the patient, and hub 19 is secured to the patient near the collarbone, using a strip of adhesive tape and/or by suturing a flange 25 to the patient. Then, blood from the patient is transported through one of tubing 21 or tubing 23 to a dialysis machine, and the other of tubing 21 and tubing 23 is used to return blood to the patient from the dialysis machine.

### SUMMARY OF THE INVENTION

It is an object of the present invention to provide a novel medical appliance securing device. It is also an object of the present invention to provide a novel method for securing a medical appliance.

According to one aspect of the invention, there is provided a medical appliance securing device, the medical appliance securing device comprising (a) a first base portion; (b) a first adhesive for securing the first base portion to an object, the first adhesive being positioned under and coupled to the first base portion; (c) a second base portion, the second base portion being positionable over the first base portion and fastenable to the first base portion; and (d) a strap, the strap having a first end coupled to the first base portion and a second end fastenable to the second base portion.

In another, more detailed aspect of the invention, the first base portion and the second base portion constitute two separate pieces of material.

In another, more detailed aspect of the invention, the first base portion and the second base portion constitute two parts of a single piece of material.

In another, more detailed aspect of the invention, the first base portion and the strap constitute two parts of a single piece of material.

In another, more detailed aspect of the invention, the first base portion, the second base portion, and the strap constitute three parts of a single piece of material.

In another, more detailed aspect of the invention, at least one of the first base portion, the second base portion, and the third base portion comprises a layer of a breathable fabric.

In another, more detailed aspect of the invention, each of the first base portion, the second base portion, and the third base portion comprises a layer of a breathable fabric.

In another, more detailed aspect of the invention, the second base portion is fastenable to the first base portion by complementary fasteners on the first base portion and the second base portion.

In another, more detailed aspect of the invention, the complementary fasteners comprise hook fasteners on one of the first and second base portions and loop fasteners on the other of the first and second base portions.

In another, more detailed aspect of the invention, the medical appliance securing device further comprises a release liner positioned under and coupled to the first adhesive.

In another, more detailed aspect of the invention, the medical appliance securing device further comprises a second adhesive for securing the first base portion to a medical appliance, the second adhesive being positioned over and coupled to the first base portion.

In another, more detailed aspect of the invention, the medical appliance securing device further comprises a second adhesive, the second adhesive being positioned over and coupled to the first base portion and being positioned over and coupled to the strap.

In another, more detailed aspect of the invention, the strap comprises a longitudinal member and a transverse member.

In another, more detailed aspect of the invention, the medical appliance securing device further comprises at least one foam block positioned over and coupled to the first base portion.

In another, more detailed aspect of the invention, the medical appliance securing device further comprises two spaced-apart foam blocks positioned over and coupled to the first base portion.

In another, more detailed aspect of the invention, the medical appliance securing device further comprises a low-adhesion surface positioned over and coupled to the second base portion.

According to another aspect of the invention, there is provided a medical appliance securing device, the medical appliance securing device comprising (a) a base, the base comprising a first portion and a second portion;(b) a strap, the strap comprising a T-shaped structure comprising a longitudinal portion and a transverse portion, the longitudinal portion being coupled to the first portion of the base, wherein the base and the strap constitute two parts of a single piece of material; (c) a first adhesive, the first adhesive being positioned under and coupled to the first portion of the base; (d) a second adhesive positioned over and coupled to each of the base and the strap; (e) a first set of fasteners positioned over the first portion of the base and coupled thereto by the second adhesive; and (f) a second set of fasteners, complementary to the first set of fasteners, positioned over the second portion of the base and coupled thereto by the second adhesive.

According to another aspect of the invention, there is provided a method of securing a medical appliance, such as a hemodialysis catheter to a patient, the hemodialysis catheter comprising a catheter hub, first and second lengths of tubing coupled to a first end of the catheter hub and a third length of tubing coupled to a second end of the catheter hub, the method comprising the steps of (a) providing a medical appliance securing device, the medical appliance securing device comprising (i) a first base portion, (ii) a first adhesive for securing the first base portion to an object, the first adhesive being positioned under and coupled to the first base portion, (iii) a second base portion, the second base portion being positionable over the first base portion and fastenable to the first base portion, and (iv) a strap, the strap having a first end coupled to the first base portion and a second end fastenable to the second base portion; (b) securing the first base portion to the patient using the first adhesive; (c) positioning the catheter hub of the hemodialysis catheter over the first base portion, with the strap positioned between the first and second lengths of tubing; (d) fastening the second base portion to the first base portion, thereby securing the catheter hub between the first and second base portions; and (e) fastening the second end of the strap to the second base portion.

For purposes of the present specification and claims, various relational terms like “top,” “bottom,” “proximal,” “distal,” “upper,” “lower,” “front,” and “rear” are used to describe the present invention when said invention is positioned in or viewed from a given orientation. It is to be understood that, by altering the orientation of the invention, certain relational terms may need to be adjusted accordingly.

Additional objects, as well as features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. In the drawings wherein like reference numerals represent like parts:

FIG. 1 is a top view of a conventional dialysis catheter;

FIG. 2 is a top view of a first embodiment of a medical appliance securing device constructed according to the teachings of the present invention, the medical appliance securing device being shown in a partially opened state;

FIG. 3 is a top view of the medical appliance securing device shown in FIG. 2, the medical appliance securing device being shown in a completely opened state (certain underlying components being shown in phantom);

FIG. 4 is a side view of the medical appliance securing device shown in FIG. 2, the medical appliance securing device being shown in a completely opened state;

FIG. 5 is a bottom view of the medical appliance securing device shown in FIG. 2, the medical appliance securing device being shown in a closed state with the bottom release liner being partially peeled away from the adhesively-coated base;

FIG. 6 is a flowchart, schematically illustrating one method for using the medical appliance securing device according to the teachings of the present invention;

FIGS. 7(a) through 7(h) are top views, illustrating various steps in the method of FIG. 6 (it being understood that certain components may not be shown in all views for the sake of clarity);

FIGS. 8(a) and 8(b) are top and side views, respectively, of a second embodiment of a medical appliance securing device constructed according to the teachings of the present invention, the medical appliance securing device being shown in a completely opened state (certain underlying components being shown in phantom in FIG. 8(a));

FIG. 9 is a partly exploded side view of a third embodiment of a medical appliance securing device constructed according to the teachings of the present invention;

FIGS. 10(a) and 10(b) are top and side views, respectively, of a fourth embodiment of a medical appliance securing device constructed according to the teachings of the present invention, the medical appliance securing device being shown in a completely opened state (certain underlying components being shown in phantom in FIG. 10(a));

FIG. 11 is a partly exploded side view of a fifth embodiment of a medical appliance securing device constructed according to the teachings of the present invention; and

FIG. 12 is a partly exploded side view of a sixth embodiment of a medical appliance securing device constructed according to the teachings of the present invention.

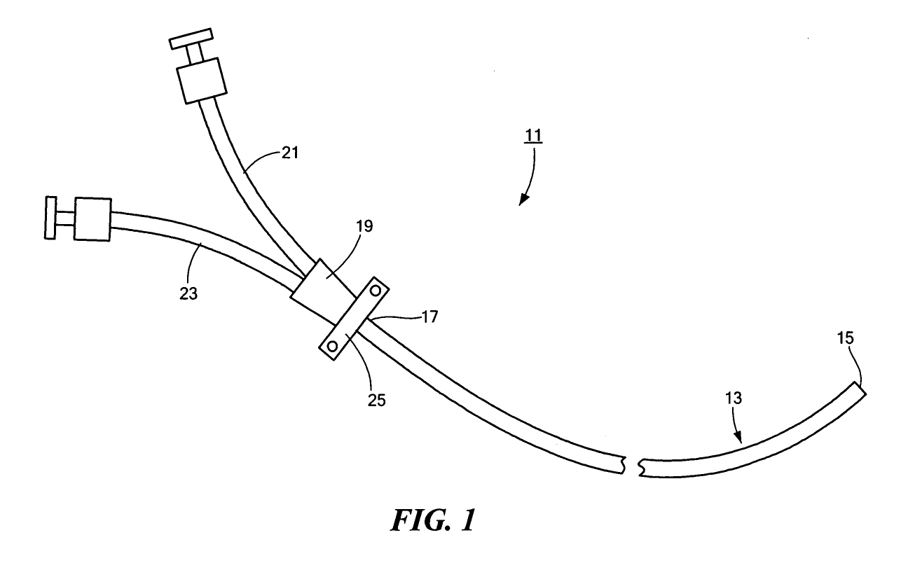

### DETAILED DESCRIPTION OF PREFERRED EMBODIMENTS

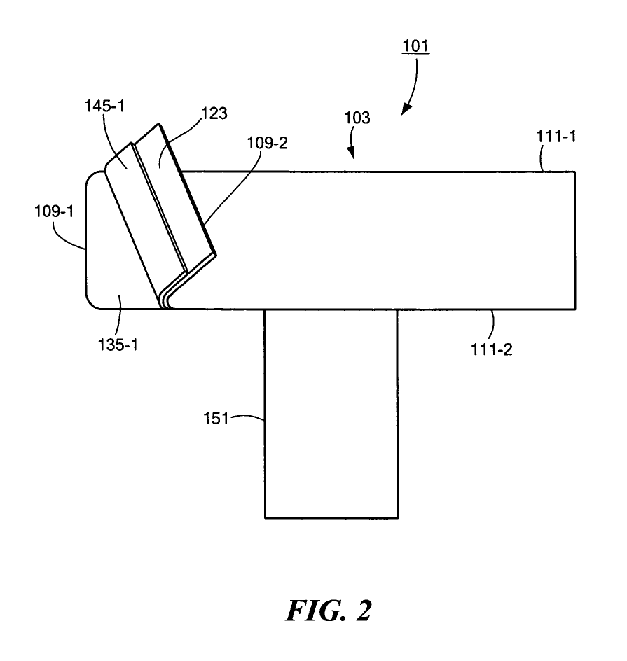

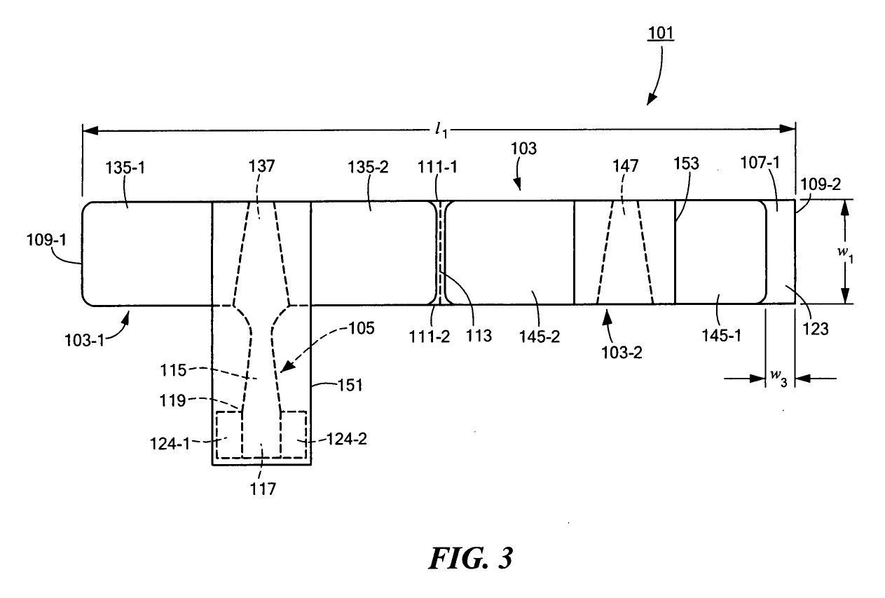

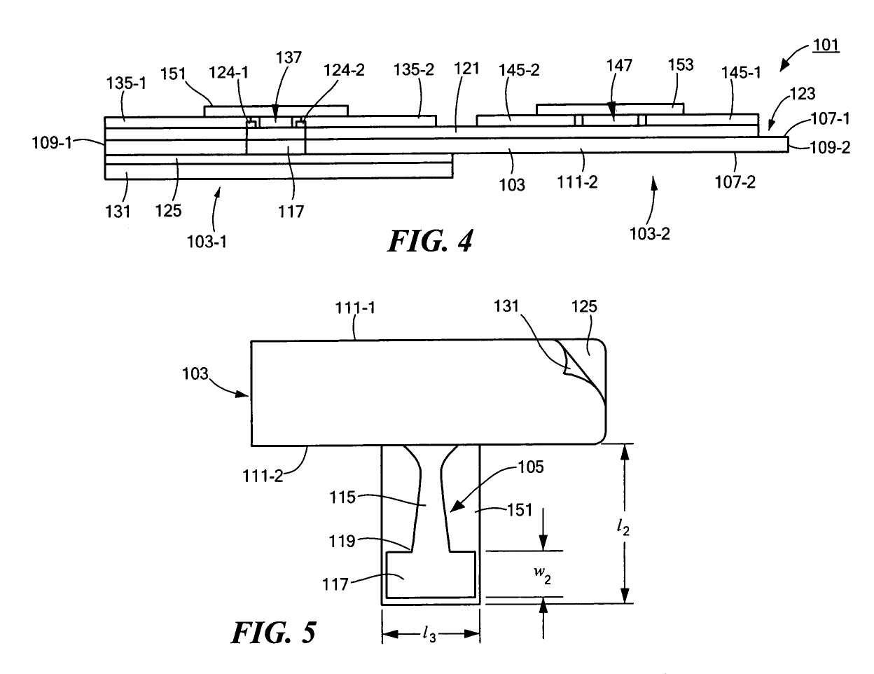

Referring now to FIGS. 2 through 5, there are shown various view of a first embodiment of a medical appliance securing device constructed according to the teachings of the present invention, said medical appliance securing device being represented generally by reference numeral 101 (it being understood that certain components of device 101 may not necessarily be shown to scale in FIGS. 2 through 5 and that, for clarity, certain components of device 101 may not be shown in one or more of FIGS. 2 through 5). In the present embodiment, device 101 is particularly well-suited for securing a hemodialysis catheter to a patient; however, device 101 is not intended to be limited to this use and may also be used to secure other types of medical devices, particularly catheters having at least one branched end.

Device 101 may comprise a unitary, i.e., one-piece, sheet of fabric shaped to define a base 103 and a flexible strap 105. The unitary sheet of fabric used to define base 103 and strap 105 may comprise a layer of breathable fabric, such as a layer of SONTARA® non-woven, spun-laced, hydro-entangled, polyester fabric having a basis weight of about 1.2 oz/y² (E.I. du Pont de Nemours and Company, Wilmington, Del.). Base 103 may be generally rectangular in shape and may include a top surface 107-1, a bottom surface 107-2, a first end 109-1, a second end 109-2, a first side 111-1, and a second side 111-2. Base 103 may include first and second portions 103-1 and 103-2, respectively, that are positioned on opposite sides of an imaginary centerline 113. Exemplary dimensions for base 103 may include a length l₁ of approximately 6.062 inches and a width w₁ of approximately 0.875 inch.

Strap 105 may be generally “T”-shaped in structure and may comprise a longitudinal member 115 and a transverse member 117. Longitudinal member 115 may extend perpendicularly from the approximate midpoint of second side 111-2 of first portion 103-1, and transverse member 117 may extend perpendicularly relative to longitudinal member 115 at an end 119 thereof distal to second side 111-2. Exemplary dimensions for strap 105 may include an overall length l₂ of approximately 1.25 inch, a length l₃ of approximately 0.813 inch for transverse member 117, a width w₂ of approximately 0.375 inch for transverse member 117, and a width for longitudinal member 115 ranging from approximately 0.188 inch proximate to second side 111-2 to approximately 0.313 proximate to transverse member 117.

Device 101 may additionally comprise an adhesive layer 121, which may be applied directly to the entirety of the top surface of strap 105 and to the entirety of top 107-1 of base layer 103, except over an area 123 of second portion 103-2 proximate to second end 109-2. (Area 123 may have a width w₃ of, for example, approximately 0.25 inch.) Adhesive layer 121 may comprise a medical grade pressure-sensitive adhesive and may be, for example, an acrylic pressure-sensitive adhesive having a thickness of about 0.001 inch. Patches 124-1 and 124-2 of a polymeric film or like material may be used to cover adhesive layer 121 at opposite ends of transverse member 117 of strap 105. In addition, although not shown, a polymeric film or like material may be applied to the bottom surface of strap 105.

Device 101 may additionally comprise an adhesive layer 125, which may be applied directly to bottom 107-2 of base layer 103 for the entirety of first portion 103-1. Adhesive layer 125 may comprise a medical grade pressure-sensitive adhesive and may be, for example, an acrylic pressure-sensitive adhesive having a thickness of about 0.001 inch.

Device 101 may further comprise a peelable release liner 131 removably adhered to the bottom of adhesive layer 125. Liner 131 may have dimensions matching that of adhesive layer 125, as in the present embodiment, or may additionally include a tab (not shown) extending away from adhesive layer 125 to facilitate liner 131 being peeled away from adhesive layer 125 by a user.

Device 101 may further comprise a pair of VELCRO® hook-type fastening patches 135-1 and 135-2 secured to top surface 107-1 of first portion 103-1 by adhesive layer 121. Patches 135-1 and 135-2, which may be generally trapezoidal in shape and which may match the borders of base 103 along three of its sides, may be appropriately dimensioned and spaced apart from one another to expose an area 137 of adhesive layer 121, area 137 having a generally trapezoidal shape that tapers from a greater width at second side 111-2 (e.g., approximately 0.628 inch) to a lesser width at first side 111-1 (e.g., approximately 0.500 inch), area 137 being aligned generally with strap 105.

Device 101 may further comprise a pair of VELCRO® loop-type fastening patches 145-1 and 145-2 secured to top surface of second portion 103-2 by adhesive layer 121, patches 145-1 and 145-2 being mateable with patches 135-1 and 135-2, respectively. (Although, in the present embodiment, patches 135-1 and 135-2 are hook-type fastening patches and patches 145-1 and 145-2 are complementary loop-type fastening patches, patches 135-1 and 135-2 could be loop-type fastening patches and patches 145-1 and 145-2 could be complementary hook-type fastening patches.) Patches 145-1 and 145-2, which may be generally trapezoidal in shape and which may match the borders of base 103 along three of its sides, may be appropriately dimensioned and spaced apart from one another to expose an area 147 of adhesive layer 121, area 147 having a generally trapezoidal shape that tapers from a greater width at second side 111-2 (e.g., approximately 0.628 inch) to a lesser width at first side 111-1 (e.g., approximately 0.500 inch). Area 147 may be appropriately positioned so as to be substantially aligned with area 137 when patches 145-1 and 145-2 are mated with patches 135-1 and 135-2, respectively.

Device 101 may further comprise a plurality of peelable release liners 151 and 153. Liner 151 may be removably adhered to area 137, as well as to the adhesively exposed portion of strap 105, so as not to expose the adhesive thereat until desired. Liner 153 may be removably adhered to area 147 so as not to expose the adhesive thereat until desired.

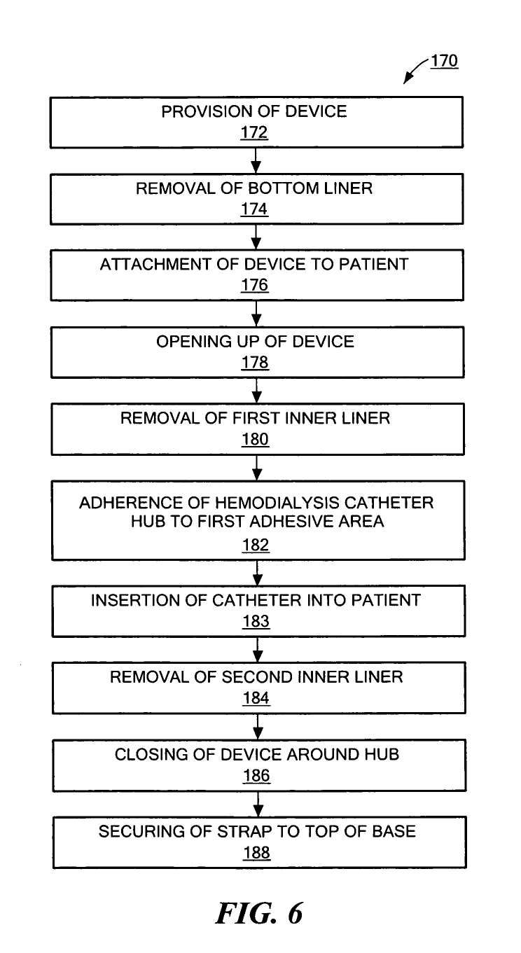

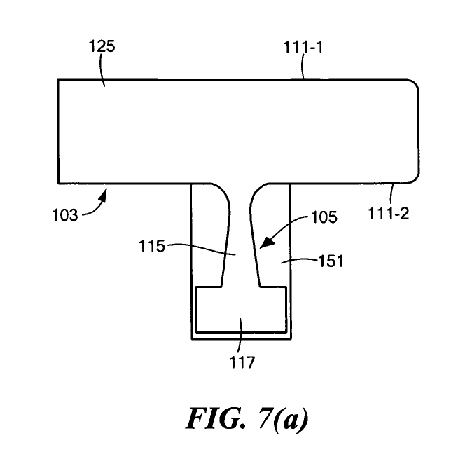

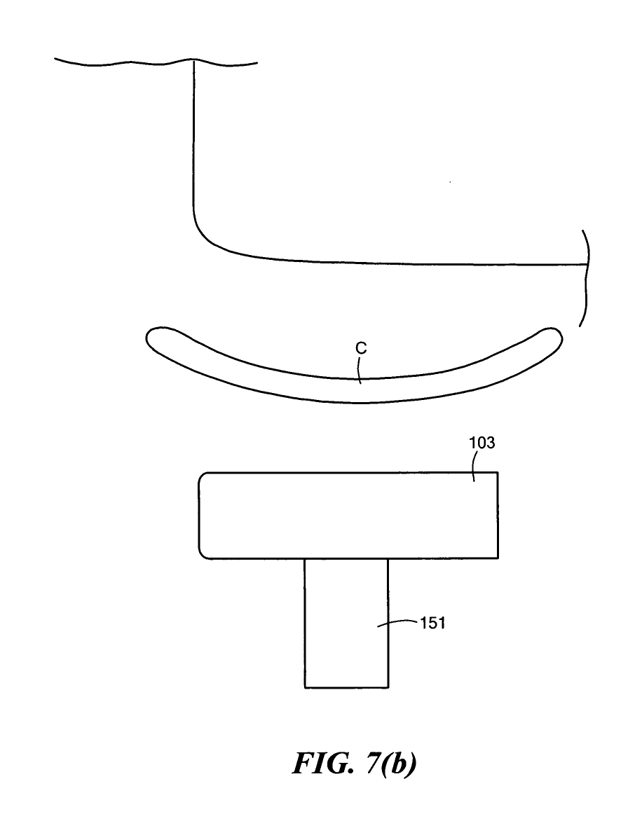

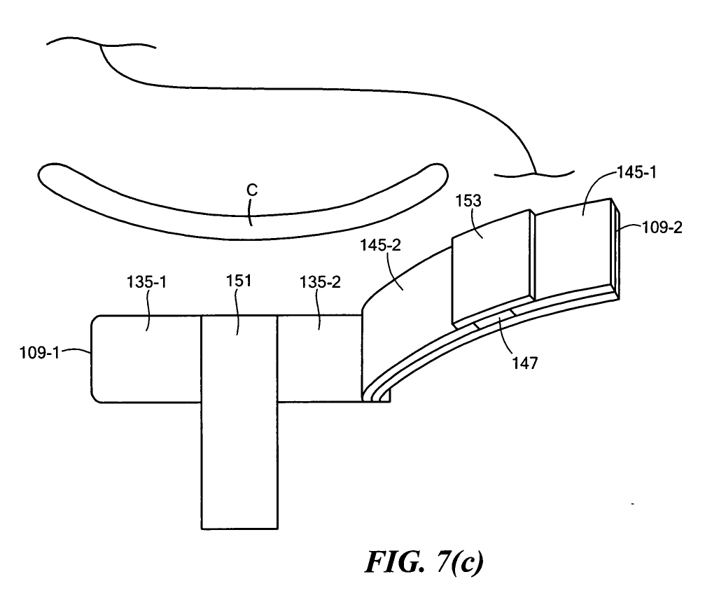

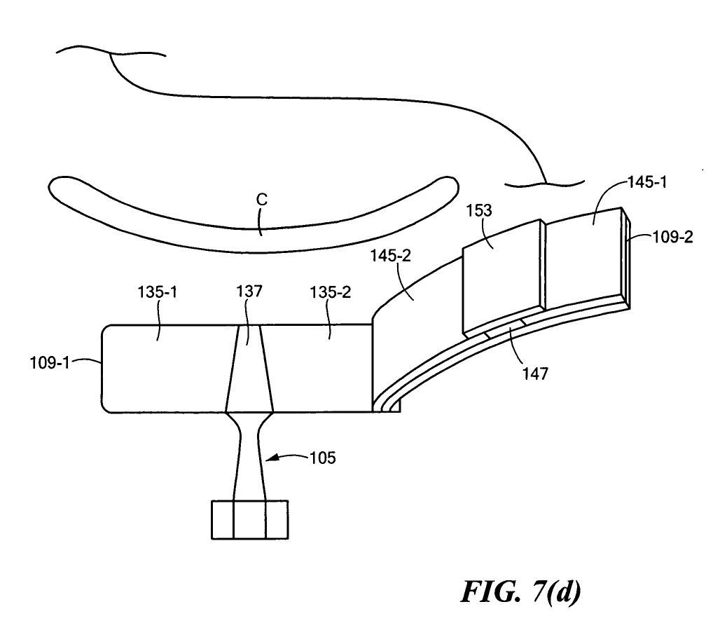

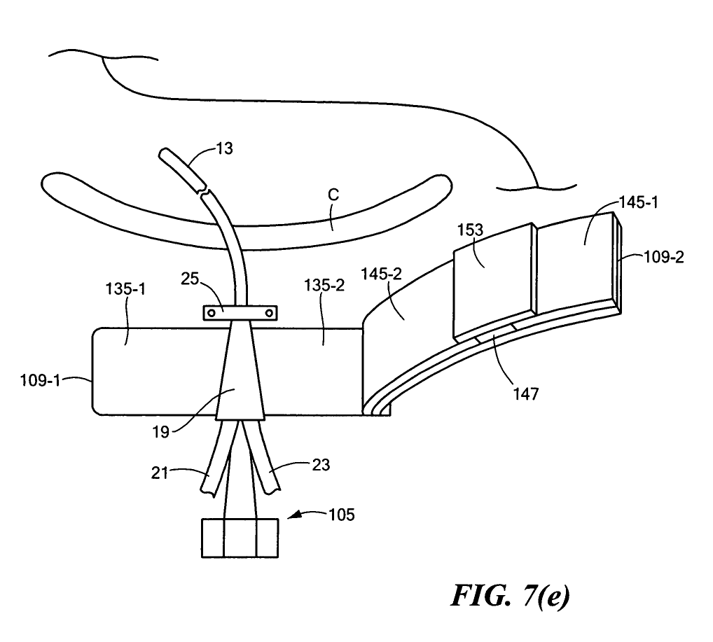

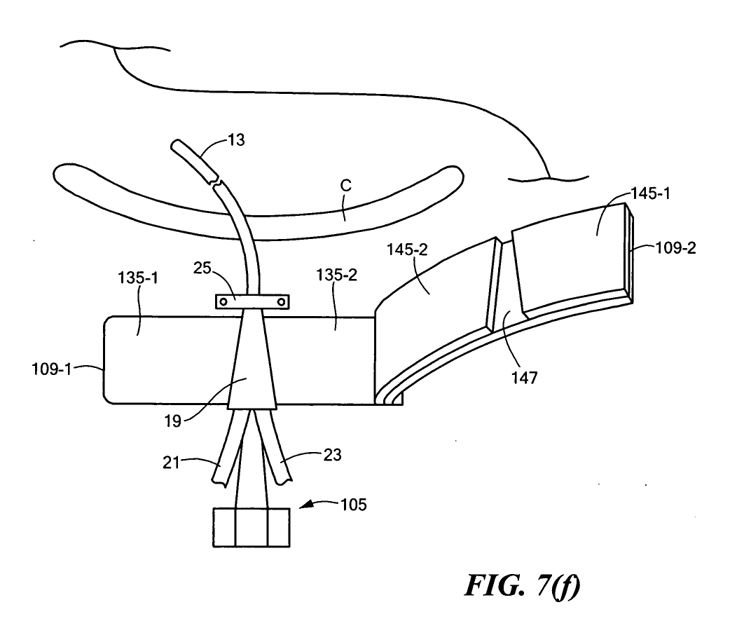

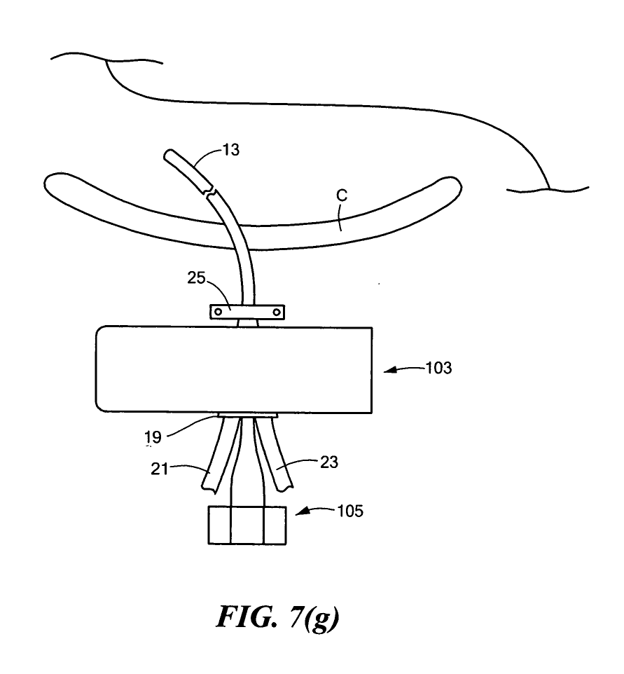

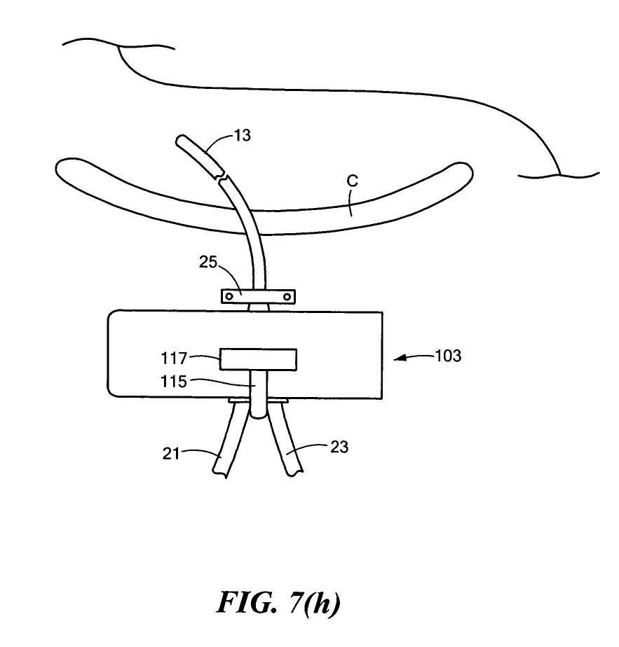

Referring now to FIG. 6, there is shown a flowchart, schematically depicting one method for using device 101 according to the present invention, the method being represented generally by reference numeral 170. (In addition, some of the steps of method 170 are also depicted in FIGS. 7(a) through 7(h).) Although the present method involves the securement of a hemodialysis catheter to a patient using device 101, it is to be understood that device 101 is not limited to securing such devices.

Method 170 can begin in a step 172 with the provision of device 101. Method can then continue in a step 174 with the removal of liner 131 (by peeling liner 131 away in the manner shown in FIG. 5) to expose the entirety of adhesive layer 125 (see FIG. 7(a)). Method 170 can then continue in a step 176 with the attachment of device 101 to a patient via adhesive layer 125. Where, as in the case of the present embodiment, device 101 is being used to secure a hemodialysis catheter to a patient, device 101 may be secured to the patient near the collarbone C of the patient (see FIG. 7(b)). Method 170 can then continue in a step 178 with the opening up of device 101 to receive a hemodialysis catheter by the pulling apart of fastening patches 145-1 and 145-2 from fastening patches 135-1 and 135-2, respectively (see FIG. 7(c)). As can be appreciated, the lack of a fastening patch in area 123 facilitates the initial pulling apart of the mated fastening patches. Also, it should be noted that because fastening patches 145-1 and 145-2 and fastening patches 135-1 and 135-2 are all positioned on a common support, namely, base 103, base 103 forms a spine-like structure when the aforementioned fastening patches are pulled away from one another. In fact, in some cases, whereas patches 135-1 and 145-1 generally do need to be completely separated from each other, it may not be necessary for patches 135-2 and 145-2 to be completely separated from each other, so long as the medical device being secured can be adequately positioned over adhesive areas 137 and 147.

Method 170 can then continue in a step 180 with the removal of liner 151, thereby exposing adhesive area 137 (see FIG. 7(d)). Method 170 can then continue in a step 182 with the adherence of the hemodialysis catheter hub 19 to adhesive area 137 (see FIG. 7(e)). Method 170 can then continue in a step 183 with the insertion of the distal end 15 of catheter 11 into the patient. Method 170 can then continue in a step 184 with the removal of liner 153, thereby exposing adhesive area 147 (see FIG. 7(f)). Method 170 can then continue in a step 186 with the closing of device 101 around hub 19 while adhering adhesive area 147 to hub 19 and re-fastening patches 135-1 and 135-2 to patches 145-1 and 145-2, respectively (see FIG. 7(g)). Method 170 can then conclude in a step 188 with the securing of strap 105 to the thus-provided top surface of portion 103-2 (see FIG. 7(h)), strap 105 providing additional restraint against the movement of hemodialysis catheter 11.

As can be appreciated, depending on the adhesive strength of adhesive layer 121, hemodialysis catheter 11 may be removed from device 101, repositioned relative thereto, and then re-adhered to device 101. As can also be appreciated, when catheter 11 is no longer needed, strap 105 may be separated from portion 103-2, device 101 may be re-opened, and catheter 11 may be removed therefrom. Device 101 may then be removed from the patient.

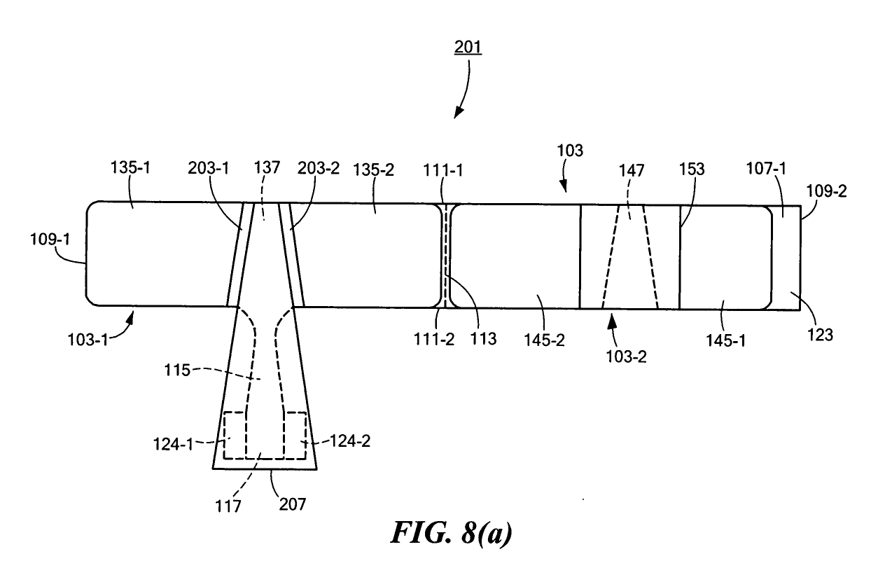

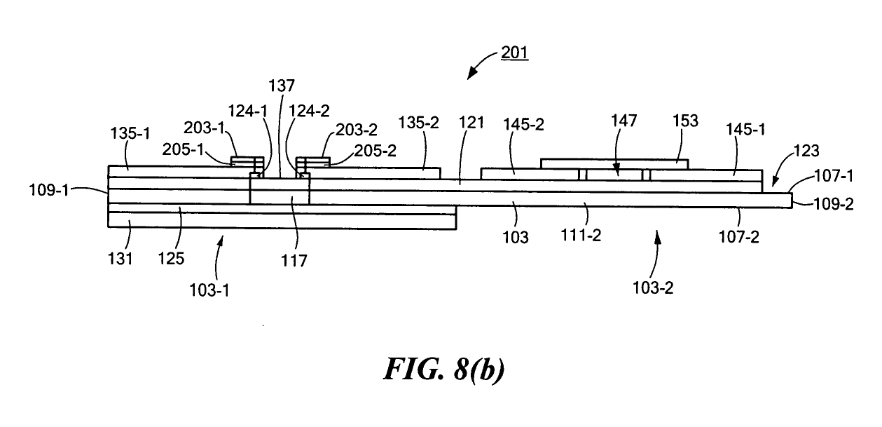

Referring now to FIGS. 8(a) and 8(b), there are shown top and side views, respectively, of a second embodiment of a medical appliance securing device constructed according to the teachings of the present invention, the medical appliance securing device being represented generally by reference numeral 201 (it being understood that certain components of device 201 may not necessarily be shown to scale and that, for clarity, certain components of device 201 may not be shown in one or both of FIGS. 8(a) and 8(b)).

Device 201 may be similar in many respects to device 101. One difference between device 201 and device 101 may be that device 201 may additionally comprise a pair of foam blocks 203-1 and 203-2. Block 203-1 may be mounted by an adhesive layer 205-1 on patch 135-1 along the edge of patch 135-1 that borders adhesive area 137. Block 203-2 may be mounted by an adhesive layer 205-2 on patch 135-2 along the edge of patch 135-2 that borders adhesive area 137. Blocks 203-1 and 203-2 may serve to provide additional restraint against rolling to a hub 19 positioned on top of area 137. (It should be understood that, whereas, in the present embodiment, blocks 203-1 and 203-2 are mounted on top of patches 135-1 and 135-2, respectively, blocks 203-1 and 203-2 could alternatively be mounted directly on adhesive layer 121 along the inside edges of patches 135-1 and 135-2, respectively.)

Another difference between device 201 and device 101 may be that, whereas device 101 may include liner 151, which may be generally rectangularly-shaped, device 201 may include a liner 207 (shown in FIG. 8(a)), which may be generally wedge-shaped.

Device 201 may be used in much the same manner as device 101.

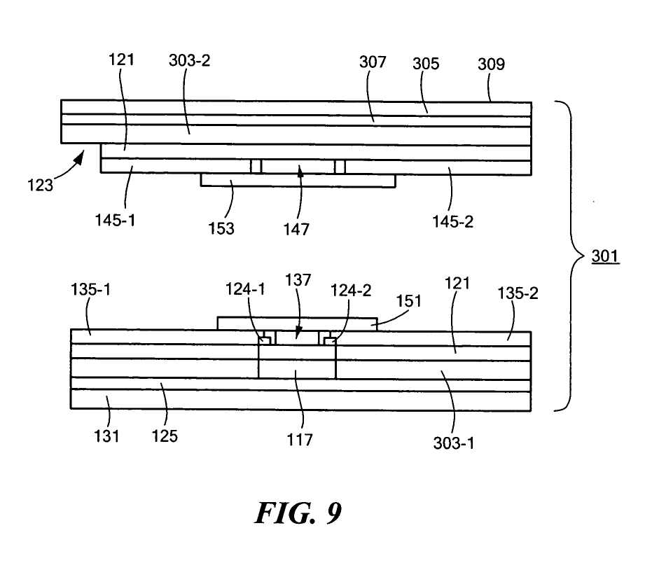

Referring now to FIG. 9, there is shown a partly exploded side view of a third embodiment of a medical appliance securing device constructed according to the teachings of the present invention, the medical appliance securing device being represented generally by reference numeral 301 (it being understood that certain components of device 301 may not necessarily be shown to scale and that, for clarity, certain components of device 301 may not be shown).

Device 301 may be similar in many respects to device 101. One difference between device 301 and device 101 may be that, whereas device 101 may comprise first and second portions 103-1 and 103-2 that are hingedly interconnected to one another about centerline 113, device 301 may alternatively comprise base portions 303-1 and 303-2 that constitute two separate pieces.

Another difference between device 301 and device 101 may be that device 301 may additionally comprise a film 305 adhered by an adhesive layer 307 to the top surface of base portion 303-2. Film 305 preferably has a low-adhesion top surface 309 so that strap 105 may be repositionably adhered to film 305.

Device 301 may be used in much the same manner as device 101.

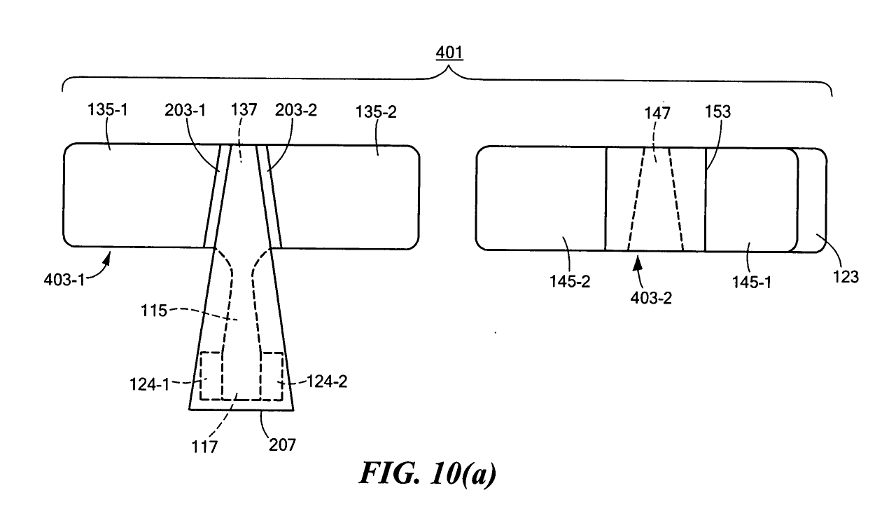

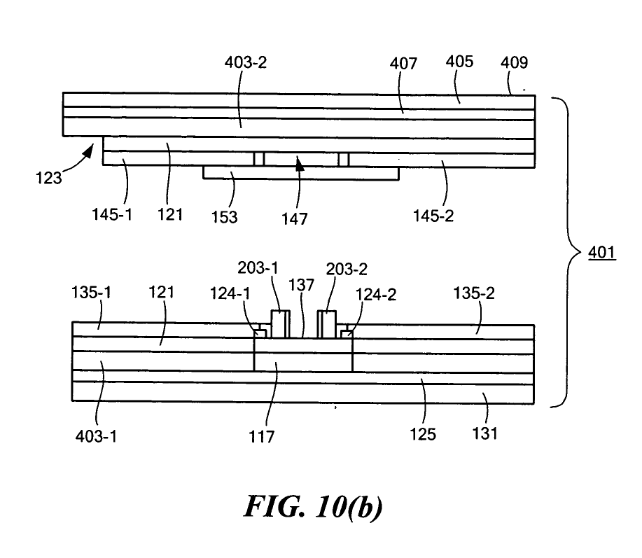

Referring now to FIGS. 10(a) and 10(b), there are shown top and side views, respectively, of a fourth embodiment of a medical appliance securing device constructed according to the teachings of the present invention, the medical appliance securing device being represented generally by reference numeral 401 (it being understood that certain components of device 401 may not necessarily be shown to scale and that, for clarity, certain components of device 401 may not be shown in one or both of FIGS. 10(a) and 10(b)).

Device 401 may be similar in many respects to device 201. One difference between device 401 and device 201 may be that, whereas device 201 may comprise first and second portions 103-1 and 103-2 that are hingedly interconnected to one another about centerline 113, device 401 may alternatively comprise base portions 403-1 and 403-2 that constitute two separate pieces.

Another difference between device 401 and device 201 may be that whereas, in device 201, blocks 203-1 and 203-2 may be adhered to patches 135-1 and 135-2, respectively, in device 401, blocks 203-1 and 203-2 may be in direct contact with adhesive layer 121.

Another difference between device 401 and device 201 may be that device 401 may additionally comprise a film 405 adhered by an adhesive layer 407 to the top surface of base portion 403-2. Film 405 preferably has a low-adhesion top surface 409 so that strap 105 may be repositionably adhered to film 405.

Device 401 may be used in much the same manner as device 101.

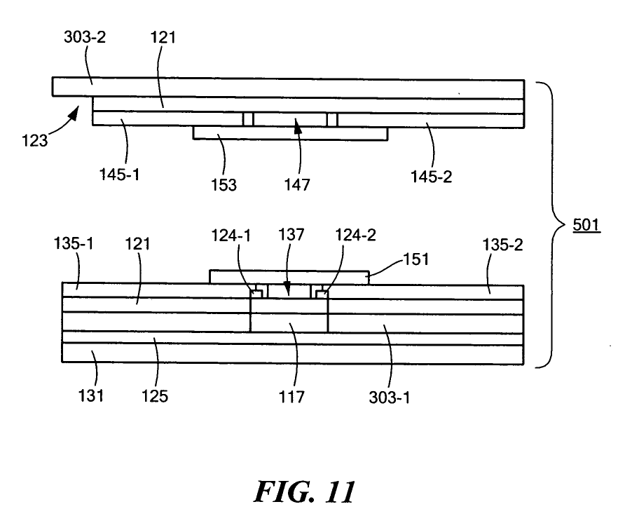

Referring now to FIG. 11, there is shown a partly exploded side view of a fifth embodiment of a medical appliance securing device constructed according to the teachings of the present invention, the medical appliance securing device being represented generally by reference numeral 501 (it being understood that certain components of device 501 may not necessarily be shown to scale and that, for clarity, certain components of device 501 may not be shown).

Device 501 may be similar in most respects to device 301, the principal difference between the two devices being that device 501 does not include structure corresponding to film 305 and adhesive layer 307 of device 301.

Device 501 may be used in much the same manner as device 101.

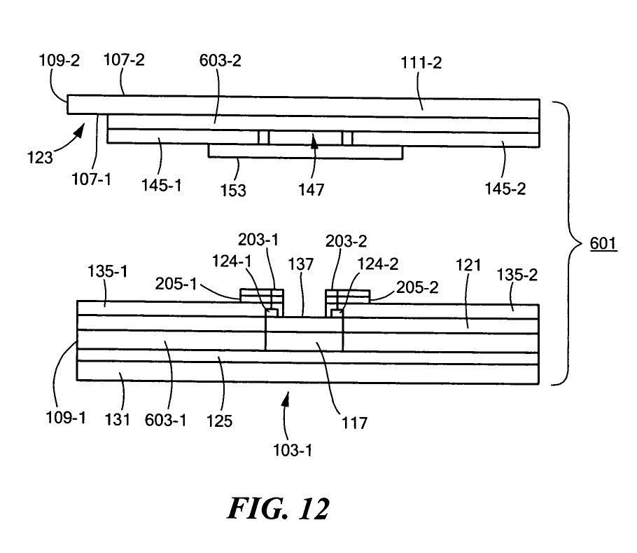

Referring now to FIG. 12, there is shown a partly exploded side view of a sixth embodiment of a medical appliance securing device constructed according to the teachings of the present invention, the medical appliance securing device being represented generally by reference numeral 601 (it being understood that certain components of device 601 may not necessarily be shown to scale and that, for clarity, certain components of device 601 may not be shown).

Device 601 may be similar in most respects to device 201, the principal difference between the two devices being that, whereas device 201 may comprise first and second portions 103-1 and 103-2 that are hingedly interconnected to one another about centerline 113, device 601 may alternatively comprise base portions 603-1 and 603-2 that constitute two separate pieces.

Device 601 may be used in much the same manner as device 101.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A medical appliance securing device comprising:
(a) a first base portion;
(b) a first adhesive for securing the first base portion to an object, the first adhesive being positioned under and coupled to the first base portion;
(c) a second base portion, the second base portion being positionable over the first base portion and fastenable to the first base portion; and
(d) a strap, the strap having a first end coupled to the first base portion and a second end fastenable to the second base portion.

2. The medical appliance securing device as claimed in claim 1 wherein the first base portion and the second base portion constitute two separate pieces of material.

3. The medical appliance securing device as claimed in claim 1 wherein the first base portion and the second base portion constitute two parts of a single piece of material.

4. The medical appliance securing device as claimed in claim 1 wherein the first base portion and the strap constitute two parts of a single piece of material.

5. The medical appliance securing device as claimed in claim 1 wherein the first base portion, the second base portion, and the strap constitute three parts of a single piece of material.

6. The medical appliance securing device as claimed in claim 1 wherein at least one of the first base portion, the second base portion, and the third base portion comprises a layer of a breathable fabric.

7. The medical appliance securing device as claimed in claim 1 wherein each of the first base portion, the second base portion, and the third base portion comprises a layer of a breathable fabric.

8. The medical appliance securing device as claimed in claim 1 wherein the second base portion is fastenable to the first base portion by complementary fasteners on the first base portion and the second base portion.

9. The medical appliance securing device as claimed in claim 8 wherein the complementary fasteners comprise hook fasteners on one of the first and second base portions and loop fasteners on the other of the first and second base portions.

10. The medical appliance securing device as claimed in claim 1 further comprising a release liner positioned under and coupled to the first adhesive.

11. The medical appliance securing device as claimed in claim 1 further comprising a second adhesive for securing the first base portion to a medical appliance, the second adhesive being positioned over and coupled to the first base portion.

12. The medical appliance securing device as claimed in claim 1 further comprising a second adhesive, the second adhesive being positioned over and coupled to the first base portion and being positioned over and coupled to the strap.

13. The medical appliance securing device as claimed in claim 1 wherein the strap comprises a longitudinal member and a transverse member.

14. The medical appliance securing device as claimed in claim 1 further comprising at least one foam block positioned over and coupled to the first base portion.

15. The medical appliance securing device as claimed in claim 1 further comprising two spaced-apart foam blocks positioned over and coupled to the first base portion.

16. The medical appliance securing device as claimed in claim 4 further comprising a low-adhesion surface positioned over and coupled to the second base portion.

17. A medical appliance securing device comprising:
(a) a base, the base comprising a first portion and a second portion;
(b) a strap, the strap comprising a T-shaped structure comprising a longitudinal portion and a transverse portion, the longitudinal portion being coupled to the first portion of the base, wherein the base and the strap constitute two parts of a single piece of material;
(c) a first adhesive, the first adhesive being positioned under and coupled to the first portion of the base;
(d) a second adhesive positioned over and coupled to each of the base and the strap;
(e) a first set of fasteners positioned over the first portion of the base and coupled thereto by the second adhesive; and
(f) a second set of fasteners, complementary to the first set of fasteners, positioned over the second portion of the base and coupled thereto by the second adhesive.

18. A method of securing a hemodialysis catheter to a patient, the hemodialysis catheter comprising a catheter hub, first and second lengths of tubing coupled to a first end of the catheter hub and a third length of tubing coupled to a second end of the catheter hub, the method comprising the steps of:
(a) providing a medical appliance securing device, the medical appliance securing device comprising
(i) a first base portion,
(ii) a first adhesive for securing the first base portion to an object, the first adhesive being positioned under and coupled to the first base portion,
(iii) a second base portion, the second base portion being positionable over the first base portion and fastenable to the first base portion, and
(iv) a strap, the strap having a first end coupled to the first base portion and a second end fastenable to the second base portion;
(b) securing the first base portion to the patient using the first adhesive;
(c) positioning the catheter hub of the hemodialysis catheter over the first base portion, with the strap positioned between the first and second lengths of tubing;
(d) fastening the second base portion to the first base portion, thereby securing the catheter hub between the first and second base portions; and
(e) fastening the second end of the strap to the second base portion.
